(19) United States
(12) Patent Application Publication
(10) Pub. No.: US 2024/0277588 A1
(43) Pub. Date: Aug. 22, 2024

(54) COSMETIC METHOD FOR HAIR WITH OZONE

(71) Applicant: Purò S.r.l., Argelato (BO) (IT)

(72) Inventors: Federico Montanari, Argelato (BO) (IT); Flavio Venturi, Argelato (BO) (IT); Flavio Garoia, Argelato (BO) (IT); Davide Marchesini, Argelato (BO) (IT)

(73) Assignee: Purò S.r.l., Argelato (BO) (IT)

(21) Appl. No.: 18/581,508

(22) Filed: Feb. 20, 2024

(30) Foreign Application Priority Data
Feb. 20, 2023 (IT) 102023000002838

(51) Int. Cl. A61K 8/22; A61Q 5/08

(52) U.S. Cl. CPC A61K 8/22; A61Q 5/08; A61K 2800/884

(57) ABSTRACT

The object of the invention relates to a cosmetic method for hair care comprising the following steps: conducting a technical treatment on the hair, comprising at least the sub-phases of applying the one or more compositions functional to the technical treatment to the hair, leaving the one or more compositions functional to the technical treatment in place on the hair, washing the hair to remove the one or more compositions functional to the technical treatment from the hair; applying ozone to damp hair for a period of time >0.5 and <21 minutes, at an ozone dosage range of ≥0.01 to ≤2000 mg/h, excluding the ozone dosage range of 180 to 400 mg/h.

This application claims priority to and the benefit of Italian Patent Application No.: 102023000002838 filed on Feb. 20, 2023, the content of which is incorporated herein by reference in its entirety.

### FIELD OF THE INVENTION

The field of the present invention relates to a cosmetic method for hair care based on ozone.

### STATE OF THE ART

Ozone therapy is an ozone-based therapy or treatment that is known to produce a number of benefits to combat a wide range of pathologies (herniated discs, acne, phlebitis, fibromyalgia, infections, hemorrhoids, arthritis, cellulite, ulcers, burns, osteoporosis). This treatment significantly improves the body's defence systems against the action of free radicals, which are mainly responsible for cellular ageing and tissue oxidation.

Ozone O₃ is a soluble gas in aqueous solution (˜13 times more than O₂ at 0-30° C.) with a solubility inversely proportional to temperature and pH. It is also characterised by a high oxidative potential (redox potential of +2.07 V vs. redox potential of the oxygen molecule O₂ of +1.23 V), which is lower than only some substances, and significantly higher than that of chlorine (+1.36 V).

It forms naturally in the human body as it is produced by white blood cells called neutrophils; thus, within the body, it performs the delicate task of counteracting the action of bacteria and fungi and, in general, all abnormal elements in the body.

In addition to its therapeutic use, ozone can also be used in cosmetics, for example for hair care.

At the state of the art, ozone is used during technical hair treatments, such as colouring, bleaching or styling, more specifically during the application period of the technical treatment or in progression thereof. Such use usually leads to advantages in terms of the duration of the colour or the blow-dry, and the intensity of hair colouring. In other words, ozone has an adjuvant or enhancing effect on the performance of the technical treatment carried out on a user's hair.

In particular, several earlier documents show that the use of ozone is functional in terms of the performance of technical treatment, e.g. in order to speed up the process of technical treatment, to make it more efficient or characterised by better results (e.g. a greater bleaching effect or a more vivid and intense hair colour).

Examples of such applications are described in the following patent documents:

FR3010305 describes a perming method comprising the following steps: rolling the hair in curlers and applying a “first perming agent” having a reducing action, in order to dilate and soften the hair; rinsing the hair to remove the first perming agent; applying to the hair an “intermediate treating/conditioning agent” (or “intermediate agent” having a “neutralising” capacity), i.e., “one or more compounds selected from the group consisting of: ‘ascorbic acid, an ascorbic acid derivative and a salt of ascorbic acid’ and ozone, in order to oxidise the intermediate agent and reconstruct the disulphide bridges; apply a ‘second permanent agent’ to the hair, when the hair is dry, to further reconstruct the disulphide bridges, in a time of between 1 and 3 minutes. In accordance with the method described in FR3010305, ozone has an oxidising function and, in this sense, together with the intermediate agent, acts as a “neutralising agent”, i.e. capable of recomposing the disulphide bonds in the hair, following the use of the first permanent agent with the opposite action, i.e. reducing, stabilising the new shape (wave) of the hair. In addition, the application of ozone takes place during the technical treatment of the perm. The authors of FR3010305 also consider that, in a humid environment, the reconstruction of the disulphide bridges proceeds in a direction that weakens the hair shape obtained during the drying phase and, when the hair dries, the wave shape is not the same as when the hair was wet. The ozone concentration according to the method of FR3010305 is between 0.5 and 1 ppm; the reason why the ozone concentration is set to a maximum value of 1 ppm is related to safety considerations with respect to ozone sprays, and the human body. As described in FR3010305, the treatment duration is between 3 and 15 minutes.

EP0103547 describes an apparatus for generating steam and ozone—ozonised steam—in controllable quantities for the treatment of both skin and hair. For hair treatment, a hair dryer (or bell) is used, especially when applying dyes or perms. Treatment with the steam/ozone mixture described in EP0103547 can enhance the bleaching process of hair keratins, and improve hair dyeing and pigmentation processes. According to the authors of EP0103547, ozone prevents itching and improves the action of keratin, helping, in combination with heat, the evaporation of ammonia contained in hair dyes and bleaching products.

GB820463 concerns a method for the improvement of hair dyeing, bleaching and/or styling processes. A steam and ozone generating apparatus is used in combination with hair treatments. The invention described in GB820463 accelerates and improves the oxidation of dyes used in hair dyeing; improves the action of lightening/bleaching products; and is useful in the neutralisation of perms.

### Problem of Known Technique

As already mentioned, well-known ozone-based cosmetic methods are intended to enhance the technical treatment by virtue of the oxidising power of O₃.

Ozone, however, precisely because of its chemical properties, is harmful to the hair if used improperly: it can lead to more or less reversible structural and functional damage to the hair, more specifically damage to the hair's protein components.

Therefore, there is a need to develop new techniques for using ozone during hair treatments, minimising the harmful effects of the molecule while exploiting its beneficial and functional properties.

### SUMMARY OF THE INVENTION

The Applicant has identified a method for cosmetic hair treatment using ozone. In particular, the cosmetic hair care method comprises the following steps:
a) conduct a technical hair treatment, comprising at least the sub-phases of
   a1) apply to the hair one or more compositions functional to the technical treatment,
   a2) leave one or more compositions functional to the technical treatment on the hair,
   (a3) washing the hair to remove one or more compositions functional to the technical treatment,
b) apply ozone to damp hair
   for a period of time >0.5 and <21 minutes,
   at an ozone dosage ranging between ≥0.01 and ≤2000 mg/h, excluding the ozone dosage ranging between 180 and 400 mg/h.

### Advantages of the Invention

The cosmetic method of the invention is not intended to accentuate or improve the result or performance of the technical treatment applied to the hair, but rather focuses on the partial or total regeneration of the primordial structure of the hair pre-treatment technique, i.e. by acting at the level of the disulphide bridges, preferably cysteine disulphide bridges of the hair. This is achieved by the use of ozone following technical parameters described in the set of claims and in the following description.

The present invention differs from common ozone-based cosmetic methods in that:
involves applying ozone to the hair at a specific dosage of ozone and within a specific time frame. Higher dosages or timescales of ozone application could compromise the constitution of the hair, e.g. by damaging or deteriorating it. From the examples given, it will be more evident that the use of O₃ over a prolonged period of time produces structural, and thus also functional, damage to the hair;
is versatile since it can be used for hair care following various technical treatments; moreover, versatility of use must also be understood from the point of view of the technical instrumentation available in beauty salons. In fact, the phase of applying ozone to the hair (b) can be done by means of different types of professional instruments such as, for example, a hair brush, a hair comb, a hair dryer (with or without diffuser), a hair straightener, a hair washer, a hair cap, a hair helmet, a hair sprayer;
provides that the step of applying ozone to the hair (b) takes place when the hair is damp. Without being bound by any theory, the Applicant submits that, in the presence of moisture or a certain percentage of water on or near the hair, the contact and adherence of ozone with the hair proteins is facilitated, thereby increasing its repairing efficacy. According to the Applicant, ozone is, in fact, able to favour the closure of the hair cuticles, increasing water retention in the hair. This increases the humidity of the hair and makes it more elastic, achieving good results in terms of hair hydration and tensile strength. In addition, the presence of water reduces the concentration of ozone in the air resulting in a less aggressive mixture, but still capable of oxidising the free sulphydryl groups SH and restoring the S—S disulphide bonds of the hair proteins;
gives good results in terms of hydration, elasticity, and resistance to pulling/breakage. The cosmetic method of the invention provides an economic advantage over treatment with molecular oxygen (O₂). Indeed, on the market, the price of oxygen concentrators is higher than that of ozone generators. The former require filters and compressors, e.g. zeolite filters, which are known to be very expensive; on the other hand, for ozone treatment, it is sufficient to have at least one ozone generator. The economic advantage reflects an advantage in terms of simplicity and ease of use of the above instrument.

### DESCRIPTION OF THE FIGURES

FIG. 1—Development of the keratin degradation parameter following ozone treatment (300 minutes).

FIG. 2—Development of the keratin degradation parameter following ozone treatment (30 minutes).

### DETAILED DESCRIPTION OF THE INVENTION

As explained above, ozone O3 has a better oxidising power than oxygen O₂. Without wishing to be bound by any theory, the Applicant considers that, rather than a direct action of ozone, the oxidising capacity of O₃ can be attributed to the release of atomic oxygen O, one of the species resulting from the decomposition of ozone. In particular, atomic oxygen O is obtained by decomposing ozone, a highly unstable molecule, according to the following reaction:

O₃+hv→O₂+O

According to the Applicant, ozone promotes the formation of disulphide bridges, preferably cysteine disulphide bridges, between keratin molecules, so as to restructure the hair and make it more resistant, hydrated and shiny; moreover, ozone is believed to improve the duration of technical treatments (bleaching, perming, etc.).

For the purposes of the present invention, hair care means any process or treatment intended to restore or reconstruct, in whole or in part, the natural or primordial structure of the hair or otherwise that with which the hair was characterised prior to the technical treatment.

It should be noted that the cosmetic method according to the ozone-based invention is not a trichological treatment, where trichological treatment means a treatment aimed at treating a condition, preferably afflicting the scalp. Examples of trichological treatment are: trichological hair loss treatment (hair that is fine, damaged and/or has a tendency to fall out and break), anti-dandruff treatment or antiseborrhoeic treatment.

Wet hair, for the purposes of the present invention, is understood to mean both hair that has been wet or freshly washed or immersed in water, i.e., that has not been towel-dried, wrung or rubbed; and hair that has been towel-dried, wrung or rubbed after wetting or washing.

### (a) Conducting a Technical Hair Treatment

As previously mentioned, the cosmetic method of hair care comprises the phase of conducting a technical treatment on the hair (a), in turn comprising at least the sub-phase of:
a1) apply to the hair one or more compositions functional to the technical treatment
a2) leave one or more compositions functional to the technical treatment on the hair
(a3) wash the hair to remove one or more of the compositions functional to the technical treatment from the hair.

According to the cosmetic method of the invention, the technical treatment is chosen from among: hair cleansing, hair reconstruction, hair conditioning, colouring, bleaching, perming and combinations of the above.

For the purposes of the invention, the natural shape or structure of the hair is determined by the arrangement of the peptide chains forming the hair, preferably bonds between cysteines, and the cross-links holding them together. These bonds are preferably chosen from the group consisting of: peptide, disulphide, salt, hydrogen bridges, and combinations of the foregoing.

Hair cleansing refers to a technical treatment aimed at washing the hair. Preferably, cleansing may take place with products that induce a modification of the natural structure of the hair, e.g. shampoo-type products, e.g. shampoos based on surfactants, e.g. relatively aggressive surfactants, and/or keratin.

Hair reconstruction is defined as a technical treatment aimed at modifying or restoring the natural structure of the hair.

Hair conditioning refers to a technical treatment with the main purpose of detangling the hair, preferably after washing or cleansing with shampoo. Conditioning products contain surfactants that create a film that envelops the hair, making it slippery and easier to comb.

Colouring refers to a technical treatment by which the natural colour of the hair is modified. Colouring includes dyes of natural or synthetic origin. Colouring treatments preferably include: temporary colouring; semi-permanent colouring; toning; permanent colouring.

Bleaching is defined as any technical treatment to lighten/bleach the hair by a few tones, by means of a chemical process (destructive oxidation of melanin). Bleached hair usually undergoes an aggressive treatment that changes its structure, making it thinner and drier.

Discolouration treatments preferably include: lightening treatments, highlights, shatush, balayage, degradé.

Mèches is a technical bleaching treatment to lighten the hair. It is suitable for those with light-coloured hair, natural or otherwise. It involves bleaching thin, fairly regular strands along the entire length.

Shatush is a technical treatment involving lightly backcombing the locks and then bleaching the escaped locks: more intense at the tips, barely noticeable near the roots.

In addition, unlike streaks, shatush cannot be resumed, i.e. the bleached lengths must be cut off in order to be able to do it a second time.

Balayage refers to a technical treatment involving the use of bleaching agents and illuminants to achieve an evenly shaded effect throughout the hair.

Degrade is a technical lightening treatment, carried out vertically, obtaining a blended effect whereby the lightening is accentuated on the lengths and tips.

According to a preferred form of the cosmetic method of the invention, the technical treatment is chosen between bleaching and perming.

Functional compositions for technical treatment” means compositions/formulations/products based on natural/semi-natural/synthetic chemicals used or applied to hair for any type of technical treatment. For example, for technical hair cleansing treatment, functional compositions may be: shampoos, preferably based on relatively aggressive surfactants and/or keratin, conditioners; for the technical hair reconstruction treatment, functional compositions may be shampoos, nourishing masks, regenerating and conditioning lotions/mousses/sprays, preferably for dry, brittle and damaged hair; for the technical hair conditioning treatment, functional compositions are conditioners, conditioners, conditioners, masks, mineral salts for the technical treatment of colouring, functional compositions are permanent colours, gradual or metallic colours, temporary colours, semi-permanent colours, vegetable colours, toning agents, highlights; for the technical treatment of bleaching, functional compositions are bleaching/bleaching formulations, preferably based on oxidising agents, such as hydrogen peroxide, and/or based on ammonia for the technical treatment of perming, functional compositions are formulations with remodelling/wetting or styling capabilities of the hair structure and/or neutralising formulations (to avoid excessive structural damage to the hair).

It should be noted that the step of washing the hair to remove the functional composition(s) from the hair involves removing or permanently eliminating all traces of said functional composition(s) from the technical treatment. Preferably, said washing step is conducted with water.

According to a preferred form of implementation, the phase of conducting a technical treatment on the hair (a) may comprise further sub-phases (an) which may be prior, subsequent or intermediate to the phases (a1)-(a3) of application, laying and rinsing of the functional composition for the technical treatment. Said further sub-phases (an) are chosen from the group consisting of:
apply curlers or hairpins or other suitable tools for shaping or styling hair to the hair,
apply a further functional composition to the hair, preferably without rinsing, i.e. without washing with water to remove/remove the composition from the hair;
apply a further functional composition to the hair, preferably with rinsing, i.e. washing with water to remove/remove the composition from the hair,
rub or wring out or dab the hair, possibly with the help of a towel

and combinations of the above.

### The Technical Treatment is Bleaching

According to a preferred form of implementation, the technical treatment is bleaching and the phase of conducting a technical treatment on the hair (a) preferably includes at least the sub-phases of:
apply to the hair a first functional composition for the technical treatment, said functional composition being a bleaching composition,
leave the bleaching composition in place on the hair,
wash the hair to remove the bleaching composition from the hair.

Preferably, the technical treatment is bleaching and the phase of conducting a technical treatment on the hair (a) comprises the sub-phases of:
a1′) apply a first functional composition to the hair, preferably a bleaching or lightening composition,
a2′) leave the first functional treatment composition on the hair, preferably for between 40 and 50 minutes,
a3′) wash the hair to remove the first functional composition of the technical treatment from the hair.

According to an optional form of realisation, bleaching comprises further sub-phases following the hair washing phase:
a4′) apply to the hair a second composition functional to the technical treatment, this composition being preferably a toning composition,
a5′) leave the second technical treatment composition on the hair, preferably for between 15 and 30 minutes,
a6′) wash the hair with water to remove the second functional composition of the technical treatment from the hair.

A toning composition is a chemical colouring composition; unlike permanent colours, which act deeper into the hair structure, a toning composition requires the coloured pigment to remain only on the surface of the hair. Toning compositions differ according to their composition and colouring intensity. Toning compositions can often dry out the hair due to the presence of alcohol in them.

Still preferably, following the hair washing sub-phase and before the ozone application phase (b), bleaching includes a further sub-phase of dabbing, wringing or rubbing the damp hair, possibly with the help of a towel.

Preferably, at the conclusion of the bleaching treatment, preferably following the sub-phase of washing the hair or dabbing, wringing or rubbing damp hair, the cosmetic method of the invention comprises the phase of applying ozone to damp hair (b).

### The Technical Treatment is Permanent

According to a preferred alternative form of implementation, the technical treatment is perming and the phase of conducting a technical treatment on the hair (a) preferably includes at least the sub-phases of:
select one or more strands of hair and roll them up into curlers,
apply to the hair a first functional composition for the technical treatment, said first functional composition being a waving composition,
leave the waving composition in place on the hair,
wash the hair to remove the wavy composition,
apply to the hair a second functional composition to the technical treatment, said second functional composition being a neutralising composition,
leave the neutralising composition on the hair,
remove the curlers and wash the hair to remove the neutralising composition.

Preferably, the technical treatment is perming, and the phase of conducting a technical treatment on hair (a) comprises the sub-phases of
a0″) select one or more strands of hair and roll them in curlers,
a1″) apply to the coiled hair a first functional composition to the technical treatment, said first functional composition being preferably a hair shape modelling product (or waving composition),
a2″) leave the first technical treatment composition on the hair, preferably for between 5 and 30 minutes,
a3″) wash the hair to remove the first functional composition of the technical treatment from the hair.

Still preferably, the technical treatment of the perm includes the following further sub-phases:
a4″) apply a second functional composition to the hair, said second functional composition being preferably a neutralising composition,
a5″) leave the second technical treatment composition on the hair,
(a6′) remove curlers or any other instrument equivalent to them from the hair,
a7″) wash the hair to remove the second, optionally the third, technical treatment functional composition from the hair.

Still preferably, following the hair washing sub-phase and before the ozone application phase (b), the perm comprises a further phase of dabbing, wringing or rubbing the damp hair.

Preferably, at the conclusion of the perming treatment, preferably following the sub-step of washing the hair or dabbing, wringing or rubbing the hair, the cosmetic method of the invention comprises the step of applying ozone to damp hair (b).

### (b) Step of Applying Ozone to Hair

Preferably, the step of applying ozone to the hair (b) involves ozone in gaseous form, mixed with water or in the form of humidified ozone (ozonised water).

For the purposes of this invention, the expression “applying ozone to hair” (b) shall preferably mean
dispense gaseous ozone directly onto the hair; alternatively,
apply a mixture of ozone and water to the hair; alternatively
apply water-soluble ozone (humidified ozone or ozonised water) to the hair.

For example, ozone in gaseous form can preferably be generated from an ozone generator device. Preferably, an ozone generator comprises at least one fan and two plates, one of which is powered to generate electrical discharges; oxygen conveyed by the fan passes between the plates. Preferably, the oxygen carried between the two plates by the fan is oxygen present in the atmosphere.

Preferably, the oxygen conveyed between the two plates is pure oxygen, i.e. with a purity of ≥98%, such as that contained in an O cylinder₂ or generated by an O concentrator₂.

### Ozone Application Time and Dosage

The phase of applying ozone to the hair (b) is conducted for or within a period of time >0.5 and <21 minutes, preferably ≥1 and <21 minutes, preferably ≥1.5 and <21 minutes, preferably ≥2 and <21 minutes, preferably ≥3 and <21 minutes, preferably >3 and ≤20 minutes, preferably >3 and <15 minutes, preferably >3 and ≤10 minutes, preferably ≥5 and ≤10 minutes, preferably ≥6 and ≤10 minutes, preferably is 10 minutes.

Excluding the ozone dosage range of 180 to 400 mg/h, the ozone dosage range is >0.01 and ≤2000 mg/h, preferably >0.05 and ≤2000 mg/h, preferably ≥0.1 and ≤2000 mg/h, preferably ≥0.5 and ≤2000 mg/h, preferably ≥1 and ≤2000 mg/h, preferably ≥2 and ≤2000 mg/h, preferably >2.7 and ≤2000 mg/h, preferably >3 and ≤2000 mg/h, preferably ≥5 and ≤2000 mg/h, preferably ≥10 and ≤2000 mg/h, preferably >15 and ≤2000 mg/h, preferably ≥20 and ≤2000 mg/h, preferably ≥23 and ≤2000 mg/h, preferably ≥25 and ≤2000 mg/h, preferably ≥27 and ≤2000 mg/h, preferably ≥30 and ≤2000 mg/h, preferably ≥40 and ≤2000 mg/h, preferably ≥50 and ≤2000 mg/h, preferably ≥60 and ≤2000 mg/h, preferably ≥70 and ≤2000 mg/h, preferably ≥80 and ≤2000 mg/h, preferably ≥90 and ≤2000 mg/h, preferably ≥100 and ≤2000 mg/h, preferably ≥120 and ≤2000 mg/h, preferably ≥130 and ≤2000 mg/h, preferably ≥140 and ≤2000 mg/h, preferably >150 and ≤2000 mg/h, preferably ≥160 and ≤2000 mg/h, preferably ≥170 and ≤2000 mg/h, preferably ≥410 and ≤2000 mg/h preferably ≥420 and ≤2000 mg/h, preferably ≥430 and ≤2000 mg/h, preferably >450 and ≤2000 mg/h, preferably ≥500 and ≤2000 mg/h, preferably ≥600 and ≤2000 mg/h, preferably ≥630 and ≤2000 mg/h, preferably ≥650 and ≤2000 mg/h, preferably ≥670 and ≤2000 mg/h, preferably ≥680 and ≤2000 mg/h, preferably ≥700 and ≤ 2000 mg/h, preferably ≥800 and ≤2000 mg/h preferably ≥900 and ≤2000 mg/h, preferably ≥1000 and ≤2000 mg/h, preferably ≥1100 and ≤2000 mg/h, preferably ≥1200 and ≤2000 mg/h, preferably ≥1300 and ≤2000 mg/h, preferably ≥1400 and ≤ 2000 mg/h, preferably ≥1500 and ≤2000 mg/h, preferably ≥1600 and ≤2000 mg/h, preferably ≥1700 and ≤2000 mg/h, preferably ≥1800 and ≤2000 mg/h, preferably >1900 and ≤2000 mg/h

According to a preferred form, excluding the ozone dosage range of 180 to 400 mg/h, preferably the ozone dosage range is between ≥0.01 and ≤170 mg/h and between ≥410 and ≤2000 mg/h, preferably the ozone dosage range is between ≥0.01 and ≤170 mg/h and between ≥650 and ≤2000 mg/h, preferably ozone dosage is between ≥0.01 and ≤170 mg/h and between ≥700 and ≤2000 mg/h, preferably ozone dosage is between ≥0.01 and ≤150 mg/h and between ≥700 and ≤2000 mg/h.

The Applicant specifies that, as long as one is within the time and dosage parameters of ozone according to the ozone application step (b) of the present invention, the restructuring effect of ozone on the hair is achieved. Thus, for the purposes of the invention, by following the time and dosage intervals of ozone application, it is possible to achieve a restructuring or reconstruction effect of the primordial structure of the hair, i.e., that which has been treated prior to the technical treatment. On the other hand, longer ozone application times risk damaging the hair, with deconstructing effects comparable to those resulting from certain technical treatments, such as bleaching or perming.

At a dosage >2000 mg/h, there is a reduction in the effectiveness and/or safety of ozone use, e.g. temporary or permanent structural damage to the hair could be induced.

Preferably within a time period preferably >0.5 and <21 minutes, the phase of applying ozone to the hair (b) requires ozone in an amount preferably between 9 mg and 700 mg, preferably between 9 mg and 500 mg, preferably between 10 and 300 mg, preferably between 10 and 200 mg, preferably between 10 and 100 mg, preferably between 10 and 80 mg, preferably 70 mg.

Excluding the range between 0.0014 L/min and 0.0031 L/min, the step of applying ozone to the hair (b) preferably provides for an ozone delivery flow between 0.001 and 10 L/min, preferably between 0.002 and 10 L/min, preferably between 0.003 and 10 L/min, preferably between 0,005 and 10 L/min, preferably between 0.005 and 8 L/min, preferably between 0.005 and 7 L/min, preferably between 0.005 and 5 L/min, preferably between 0.005 and 3 L/min, preferably between 0.005 and 1 L/min, preferably between 0.01 and 5 L/min, preferably between 0.1 and 5 L/min, preferably between 0.5 and 5 L/min. Still preferably, except for the range of 0.0014 L/min to 0.0031 L/min, the step of applying ozone to the hair (b) comprises an ozone delivery flow rate of between 0.5 and 10 L/min, preferably between 1 and 10 L/min, preferably between 3 and 10 L/min, preferably between 5 and 10 L/min, preferably between 7 and 10 L/min.

The delivery pressure of ozone in gaseous form is preferably atmospheric.

### Ozone Application Step (b) on Damp Hair

For the purposes of this finding, the ozone application step (b) is conducted on essentially damp hair. Moist hair is defined as
the hair is towel-dried, optionally rubbed or wrung out, using a cloth or towel, after wetting the hair or after washing, in order to remove excess water, thus preventing the hair from dripping when excessively wet; or
the hair wet, i.e. after washing or just after wetting the hair, partially or totally; or
the hair immersed in water.

### Ozone Application Phase (b) at the Conclusion of a Technical Hair Treatment (a)

Preferably, the step of applying ozone to wet hair (b) takes place at the conclusion of the step of conducting a technical treatment on the hair (a), i.e. after the last sub-step constituting the technical treatment (a); according to a preferred embodiment, the step of applying ozone (b) is conducted on the wet hair after rinsing or washing or cleansing thoroughly with water to remove all traces of the composition(s) functional to the technical treatment.

According to the Applicant, this allows good results to be achieved in terms of hair care, since the phase of applying ozone to the hair (b) preferably takes place at the conclusion of the technical treatment, in other words following or subsequent to the completed technical treatment; without wishing to be bound by any theory, the Applicant considers that preferably the use of ozone at the conclusion of the technical treatment allows better restructuring and regeneration of the hair.

### Ozone Application Handpieces

The step of applying ozone to the hair (b) is preferably done with a handpiece chosen from the group consisting of: a hairbrush, a hair comb, a hair dryer—preferably with or without a diffuser—a hair straightener, a hair washer, a hair cap, a hair helmet, a hair sprayer.

### First Mode of Ozone Application

According to a preferred form of implementation, the phase of applying ozone to the hair (b) preferably comprises the following sub-phases:
(b1) prepare ozone in gaseous form;
(b2) prepare water in liquid form;
b3) dispense ozone in gaseous form and, at the same time, spray water in liquid form on the hair.

For the purposes of the present invention, the nebulisation of water is preferably carried out by means of a nebuliser, i.e. a device that transforms a liquid into an aerosol of very fine droplets; the liquid, injected by a nozzle, is dispersed by a diffuser.

The phase of dispensing ozone in gaseous form and, at the same time, spraying water in liquid form on the hair (b3) preferably involves dispensing ozone in gaseous form on the hair and, at the same time, spraying liquid water on the damp hair.

Preferably, the step of applying ozone to the hair (b) according to this mode is done by dispensing ozone in gaseous form at a dosage between 50 and 1000 mg/h, preferably between 50 and 800 mg/h, preferably between 50 and 500 mg/h, preferably between 50 and 170 mg/h, preferably between 50 and 160 mg/h, preferably between 50 and 150 mg/h, preferably between 60 and 130 mg/h, preferably between 70 and 100 mg/h, excluding the interval between 180 and 400 mg/h.

### Second Method of Ozone Application

In a second alternative form of implementation to the previous one, the step of applying ozone to the hair (b) preferably comprises the following sub-steps:
b1′) prepare ozone in gaseous form;
b2′) dispense ozone in gaseous form on damp hair.

It should be noted that, according to this second form of realisation and unlike the first method of applying ozone, no water in nebulised liquid form is used. Instead, the moisture already present on the damp hair is exploited.

Preferably, the step of applying or dispensing ozone on the hair (b) according to this modality is done by dispensing ozone in gaseous form at a dosage between 410 mg and 1500 mg in one hour (h) (extremes included), preferably between 450 and 1400 mg/h, preferably between 500 and 1300 mg/h, preferably between 800 and 1200 mg/h, excluding the range between 180 and 400 mg/h.

### Third Mode of Ozone Application

A third embodiment of the invention provides that the step of applying ozone to hair (b) preferably comprises the following sub-steps:
b1″) prepare ozone in gaseous form
b2″) prepare water in liquid form,
b3″) solubilise ozone in gaseous form in water to obtain humidified ozone (or ozonated water), and
b4″) apply humidified ozone to damp hair.

Preferably, the step of solubilising the ozone in gaseous form in water to obtain humidified ozone (or ozonised water) (b3″) involves the use of a bubbler device, known to the practitioner, for solubilising the ozone in gaseous form in water. In other words, the gaseous ozone is humidified, by means of a bubbler, before being applied to the hair.

A further way of carrying out the ozone solubilisation phase in gaseous form in water to obtain humidified ozone (or ozone humidification phase) (b3″) preferably involves mixing or solubilising ozone in gaseous form directly in water, preferably the water from a sink or wash basin. In this way, gaseous ozone is ‘immersed’ (solubilised) in water before being applied to the hair.

Preferably, the passage of the ozone stream through water allows the gas to dissolve, so that the microdroplets of humidified ozone (or ozonised water) adhere more effectively to the hair, improving its efficacy. For the purposes of the present invention, preferably the bubbler has a volume ≥350 ml and ≤500 ml, preferably equal to 500 ml.

For the purposes of this finding, “applying humidified ozone to wet hair” (b4″) means applying humidified or water-solubilised ozone (humidified ozone or ozonised water) to wet hair.

Preferably, the step of applying humidified ozone (or ozonised water) to the hair (b4″) takes place by nebulisation (“vaporisation”) of the humidified ozone, i.e. by means of a nebuliser or vaporiser; according to another embodiment, the step of applying humidified ozone to the hair (b4″) takes place preferably by pouring (or outflowing) at least a part of the aforementioned humidified ozone (or ozonised water) onto the already wet hair of a user.

In an alternative form of implementation, the method may involve the use of a catalyst that helps to improve the effectiveness of the treatment by recovering chemical species produced during ozone application that would be lost to the environment if not recovered.

Preferably, the step of applying or dispensing ozone on the hair (b) according to the third modality takes place preferably by applying ozone in gaseous form at a dosage between 410 mg and 1500 mg in one hour (h) (extremes included), preferably between 450 and 1400 mg/h, preferably between 500 and 1300 mg/h, preferably between 800 and 1200 mg/h, excluding the range between 180 and 400 mg/h.

According to the third ozone application method, considering the solubility of ozone in gaseous form in water at a temperature preferably between 20 and 30° C., the ozone titre (or concentration) in humidified ozone (or ozonised water) is preferably between 350 mg and 600 mg, preferably between 400 mg and 570 mg per litre of humidified ozone (or ozonised water).

Preferably, following the step of applying ozone to the hair (b), respecting the timing for the purposes of the invention, the hair may be dried, preferably by means of an ordinary hair dryer.

### EXAMPLES

Below, the Applicant gives examples of the invention for illustrative and non-limiting purposes only.

### Example 1—Study of the Effect of Ozone Treatment on Standard Bleached Hair

This study uses an ozone generator at 1000 mg/h. The study was conducted by treating standard bleached hair with ozonised air (containing electrophilic oxygen), using an ozone generator at 1000 mg/h, monitoring the ozone concentration, temperature and humidity of the treatment environment.

Ozone-containing air was used either directly (“O₃ direct”), or after being passed through a bubbler (“O₃ bubbled”) into water. The operating conditions are listed in table 1 below:

TABLE 1

| Experiment | Temperature | Relative Humidity |
|---|---|---|
| 1000 mg/h O₃ direct | 18-20° C. | 52-60% |
| 1000 mg/h O₃ bubbled | 18-20° C. | 70-75% |

The final state of the hair was determined by measuring the degradation of cysteine by monitoring the ratio of the areas of the FT-IR signals related to cysteic acid (1240 cm⁻¹), which represents the degraded cysteine, to the chain amide signal (1630-1640 cm⁻¹), which represents the total amount of protein. Table 2 below shows the keratin degradation index value as a function of time over 300 minutes of electrophilic oxygen treatment.

TABLE 2

| Time (minutes) | 1000 mg/h O₃ direct | 1000 mg/h O₃ bubbled |
|---|---|---|
| 0 | 0.34 | 0.34 |
| 2 | 0.16 | 0.20 |
| 5 | 0.15 | 0.18 |
| 7 | 0.15 | 0.16 |
| 10 | 0.20 | 0.19 |
| 15 | 0.25 | 0.18 |
| 20 | 0.27 | 0.19 |
| 30 | 0.32 | 0.25 |
| 60 | 0.35 | 0.28 |
| 120 | 0.37 | 0.32 |
| 180 | 0.40 | 0.35 |
| 240 | 0.41 | 0.36 |
| 300 | 0.40 | 0.36 |

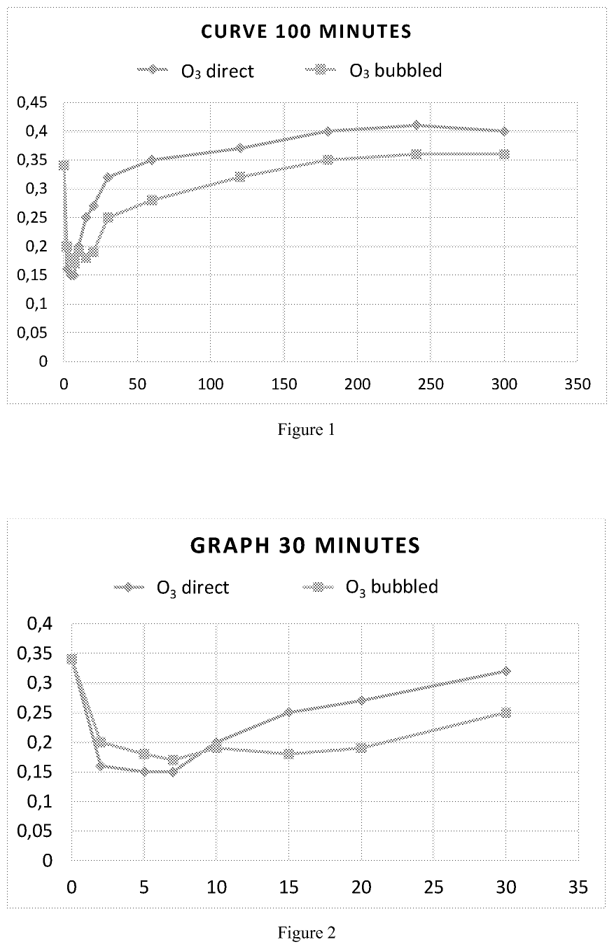

FIGS. 1 and 2 show the same data in graphical format, highlighting the trend up to 300 minutes and that in the first 30 minutes.

### Example 2—Optimisation of Parameters for Ozone Treatment

The aim of the study is to optimise the parameters for ozone treatment on bleached hair. Samples of 10 g of standard hair decolourised for 15 minutes with 30-volume hydrogen peroxide (9% in weight) and subsequently treated with ozone at different flow rates and times (always remaining within the claimed time and dosage intervals of ozone) were used. The controlled parameter is the IR degradation index of keratin, calculated from the infrared spectrum signals related to keratin degradation at 1041 cm⁻¹. The following equation (1) was developed to calculate the optimal treatment time:

$$Optimum time = 21 - 6 \cdot F$$

where F is the flow measured in L/min; time is measured in minutes.

It is possible to calculate a percentage hair regeneration index (R), understood as the ratio of the difference between the initial (I(initial)) and the current (I(current)) degradation index, divided by the initial (I(initial)) percent, as equation (2) below:

$$R = \frac{I ( iniziale ) - I ( attuale )}{I ( iniziale )} \cdot 100$$

For this hair sample I(initial) is 0.45 and is reduced to a maximum of 0.15.

By mathematically processing the trend, we obtain the following empirical equation (3), valid in the range 0-20 minutes:

$$R = \frac{67 \cdot t}{21 - 6 \cdot F} ( 2 - \frac{t}{21 - 6 \cdot F} )$$

1. Cosmetic hair care method comprising the following steps:
a) conducting a technical hair treatment, comprising at least the sub-phases of
a1) applying to the hair one or more compositions functional to the technical treatment,
a2) leaving one or more compositions functional to the technical treatment in place on the hair,
(a3) washing the hair to remove one or more compositions functional to the technical treatment,
b) applying ozone to damp hair
for a period of time >0.5 and <21 minutes,
at an ozone dosage ranging between ≥0.01 and ≤2000 mg/h, excluding the ozone dosage ranging between 180 and 400 mg/h.

2. Cosmetic method according to claim 1, in which the ozone dosage is ≥0.05 mg/h and ≤2000 mg/h.

3. Cosmetic method according to claim 1, wherein the step of applying ozone to damp hair (b) occurs at the conclusion of the step of conducting a technical treatment on the hair (a).

4. Cosmetic method according to claim 1, wherein the technical treatment is selected from the group consisting of: hair cleansing, hair reconstruction, hair conditioning, colouring, bleaching, perming and combinations thereof.

5. Cosmetic method according to claim 1, wherein the technical treatment is selected from the group consisting of bleaching and perming.

6. Cosmetic method according to claim 1, wherein the step of applying ozone to the hair (b) comprises the following sub-steps:
b1) preparing ozone in gaseous form;
b2) preparing water in liquid form;
b3) dispensing ozone in gaseous form on the hair and, at the same time, spray water in liquid form on damp hair;
or
b1′) preparing ozone in gaseous form;
b2′) dispensing ozone in gaseous form on damp hair.

7. Cosmetic method according to claim 1, wherein the step of applying ozone to the hair (b) comprises the following sub-steps:
b1″) preparing ozone in gaseous form
b2″) preparing water in liquid form,
b3″) solubilising ozone in gaseous form in water to obtain humidified ozone, and
b4″) applying humidified ozone to damp hair.

8. Cosmetic method according to claim 7, wherein the ozone titre in the humidified ozone is comprised between 350 mg and 600 mg per litre of humidified ozone.

9. A cosmetic method according to claim 1, wherein the step of applying ozone to the hair (b) is performed by a means selected from the group consisting of: a hair brush, a hair comb, a hair dryer (with or without diffuser), a hair straightener, a hair washer, a hair cap, a hair helmet, a nebulizer.

10. Cosmetic method according to claim 1, wherein the step of applying ozone to the hair (b) includes a dispensing flow rate comprised between 0.001 and 10 L/min.
